(19) United States
(12) Patent Application Publication
(10) Pub. No.: US 2008/0098498 A1
(43) Pub. Date: Apr. 24, 2008

(54) BROWN MIDRIB SUDANGRASS HYBRID 'CW 2-43-6'

(76) Inventor: Jonathan M. Reich, Woodland, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/962,437

(22) Filed: Dec. 21, 2007

Related U.S. Application Data
(62) Division of application No. 11/265,201, filed on Nov. 3, 2005.

(51) Int. Cl. A01H 5/00

(52) U.S. Cl. 800/320

(57) ABSTRACT

The invention provides sudangrass inbred and hybrid plants having adaptation, productivity, and disease resistance with reduced lignin concentration, reduced cell wall concentration, and improved digestibility. Plants and plant parts of the invention are useful in the efficient production of meat and milk due to improved whole plant and fiber digestibility.

### FIELD OF THE INVENTION

This invention relates to the field of sudangrass plants, and more specifically to improved sudangrass plants having increased levels of forage quality and methods for producing such plants.

### BACKGROUND OF THE INVENTION

All publications and patent applications herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

Sorghum is a genus of about 20 species of grasses, native to tropical and subtropical regions of the Old World, with one species native to the New World in Mexico. The genus Sorghum includes three principal distinct morphotypes that are used as forages: forage sorghums, sudangrass, and sorghum× sudangrass hybrids. These three morphotypes have grossly different phenotypes and different modes of principal utilization. Forage sorghums have very coarse stems and wide leaves, similar to corn (Zea mays L.), very low tillering capacity, and very slow speed of regrowth after cutting. Consequently they are used nearly exclusively as a silage crop, never for hay production and only occasionally as direct pasture. Sudangrass in comparison is very grassy, characterized by very fine stems and narrow leaf blades, profuse tiller development, and exceptionally rapid recovery after cutting or grazing.

Sorghum is mainly self-fertilized with natural cross pollination ranging from 2 to 35% and averaging about 6%. Wind and convection currents are the principal means of pollen movement. The inflorescence of Sorghum is a panicle that varies morphologically from compact to open. The spikelets are usually in pairs on the branches, one being sessile and fertile and the other being pedicelled and male or sterile. The terminal sessile spikelet of each branch has two pedicelled spikelet s associated with it (Shertz and Dalton, 1980).

Sorghum (Sorghum biocolor (L.) Moench) is a major food and feed grain crop and its vegetative parts are used as forage, syrup and shelter. Cultivated sorghum consists of several morphologically distinct races which readily cross. Several grassy types, e.g., S. arundinaceum, S. verticillifolium, and S. aethiopicum with the same chromosome number as S. bicolor (2N=20) can be crossed with S. bicolor with only slight barriers to gene exchange.

Sudangrass (Sorghum bicolor ssp. sudanense L.) is an important forage species for pasture, grazing, green chop silage, hay and seed. Sudangrass is also referred to by the scientific name sorghum× drummondii (Steudel) Millsp. & Chase (=S. bicolor×S. arundinaceum) (R. F. Barnes and J. B. Beard (ed.), Glossary of Crop Science Terms, Crop Science Society of America, July 1992, pg. 84). Classification and species relationships of sorghum and sudangrass are discussed in several reports (Harlan and deWet, 1972; Celarier, 1958). For a comprehensive review of the floral characteristics, plant culture, and methods of self-pollinating or hybridizing sudangrass, see Shertz and Dalton, Sorghum 41:577-588, In Hybridization of Crop Plants, Fehr et al. (ed.), American Society of Agronomy Inc. (1980).

Sorghum× sudangrass hybrids (Sorghum bicolor×S. bicolor spp. sudanese) which result from crossing a sorghum female with a sudangrass male are generally intermediate in character expression between sorghum and sudangrass. Sorghum× sudangrass hybrids are also commonly referred to as sorghum-sudangrass hybrids, sorghum/sudangrass, sudax, and sudex, (Sudax® is a registered trademark). Adding somewhat to the confusion of the nomenclature, those skilled in the art sometimes refer to sorghum× sudangrass hybrids as “sudangrass hybrids”. See, e.g., Miller and Stroup, 2003. As used herein, however, a “sudangrass hybrid” refers to a seed, cell, whole plant, plant part (e.g., root, stem, leaf, pollen, ovule, etc.), and/or tissue culture produced from or originating from the cross of two genetically different sudangrass (Sorghum bicolor ssp. sudanense L.) parental plants, wherein one parent plant is used as the female parent plant and the other parent plant is used as the male parent plant.

A comparison of the phenotypic/morphological characteristics of sudangrass, forage sorghum and sorghum× sudangrass hybrids are provided in Table 1.

TABLE 1
Characteristics of sudangrass, forage sorghum, and sorghum x sudangrass
hybrids, principal morphotypes of forage crops within the genus Sorghum.
All measurements are provided as average ranges.

| Characteristic | Sudangrass | Sorghum x sudangrass | Forage Sorghum |
|---|---|---|---|
| Stem diameter | 0.25-0.375 inches | 0.50-1.00 inches | 1.50-2.50 inches |
| Leaf Width | 0.75-1.00 inches | 1.00-1.50 inches | 1.50-2.50 inches |
| Tillering capacity | Very High | Medium | Very Low |
| Regrowth potential | Very High | Medium | Very Low |
| Adaptation for hay | Excellent | Fair | Very Poor |
| Adaptation for pasture | Excellent | Very Good | Poor |
| Adaptation for silage | Good | Excellent | Excellent |

Sudangrass is recognized as an important summer annual grass, as being capable of maintaining productivity under hot and dry conditions, useful in the improvement of soil tilth, as an important source of emergency forage, and with the capacity to reduce nematode and disease incidence of subsequent rotation crops. Sudangrass is used as a pasture crop for dairy and beef cows, sheep and hogs, and as a range plant for poultry, especially turkeys (Armah-Agyeman et al., 2002).

Although sudangrass originated in Africa, it is well adapted to a wide range of climates and soils in the United States. It was introduced into the United States in 1909 and is now one of the most valuable summer annual forage grasses. It is widely adapted, has excellent drought resistance and heat tolerance, grows rapidly, and is responsive to fertilizer and water. As a rotation crop for onions, sudangrass has improved yield and quality, decreased pest pressure, and reduced pesticide use (Cornell Univ. Report, 1997). As a green manure crop in Russet Burbank potato rotations, sudangrass has been shown to reduce incidence of Verticillium wilt, increase yield of #1 tubers, and yield of #1 tubers >280 grams (Davis et al., 2004). As a rotation crop for head lettuce, sudangrass has improved weight of lettuce heads and decreased reproduction of Meloidogyne hapla (Viaene and Abawi, 1998).

Of the Sorghum species grown for forage, sudangrass has the finest stems, tillers most profusely, and has the most rapid regrowth following cutting or grazing. The finer stems give it better drying characteristics than other Sorghums for hay making (Undersander, 2000). The fine stems, extensive tillering, and rapid regrowth of sudangrass make it better suited to pasturing than other types of Sorghum (Anderson and Guyer, 1986; Leep, 2005). Sudangrass and sorghum× sudangrass hybrids are widely grown commercially for direct pasture, hay, haylage, greenchop, and silage.

Sudangrass forage can be utilized in the immediate term by ruminant animals through direct consumption by means of grazing, or via greenchop and confined feeding. All other means of forage utilization require preservation in the form of hay, haylage, or silage, wherein such forms are referred to herein as “preserved forage”. Sudangrass pastures are commonly utilized by a system of intensive rotational grazing. Stocking rates of 2-8 head of cattle (Bos taurus) weighing 400-1000 pounds each per acre are commonly used, based on dry matter availability. Confined feeding of beef and dairy cows is a common practice with forage harvested by means of a flail harvester or chopper and the greenchop transported from the production field to the location of feeding.

Hay is the most common form of preserved sudangrass. Hay is stored at a moisture level so low that biological processes do not proceed rapidly enough to build up heat to combustion temperatures. A standing crop of sudangrass is cut with a swather, that may include crimping rollers to hasten drying, and placed in a swath or windrow in the field. The swath is raked after a few to several days, depending on drying conditions, to turn over the windrow and expose the underside. When the forage dry matter concentration is approximately 85%, the windrows can be safely baled for long term storage. Sudangrass hay is fed throughout the fall, winter, and spring as supplemental feed when fresh forage is limiting.

Sudangrass silage is the product of a controlled anaerobic fermentation of greenchopped sudangrass forage by inoculating with bacterial inoculant and storing the inoculated forage in an anaerobic environment in some type of silo including but not limited to the following: upright silo, pit silo, bunker silo, trench silo, or silage bag. Haylage is produced by inoculating partially wilted forage with bacterial inoculant and storing the inoculated forage in an anaerobic environment in some type of silo. The moisture content of partially wilted forage prior to inoculation is 35-60%. Both haylage and silage are used as supplemental feeds throughout the fall, winter, and spring when fresh forage is limiting.

Direct consumption of sudangrass pasture and confined feeding of greenchop sudangrass forage to sheep (Ovis aires) also occurs commonly. Preserved sudangrass forage in the form of hay, haylage, and silage is also commonly fed to sheep.

Commercial sudangrass seed may be provided either in an open pollinated variety or a hybrid variety. Commercial production of open pollinated varieties may include a breeder seed production stage, a foundation seed production stage, a registered seed production stage and a certified seed production stage. Hybrid variety seed production may involve up to three stages including a breeder seed production stage, a foundation seed production stage and a certified seed production stage.

Efforts in developing healthy and productive sudangrass varieties often focus on breeding for disease and stress-resistant cultivars, for example, breeding for adaptation to specific environments, breeding for yield per se, and breeding for forage quality. Success has been attained in breeding for resistance to fungal, bacterial, viral, insect, and nematode pests, including, but not limited to the development of varieties tolerant/resistant to anthracnose, downy mildew and rust. Breeders have had less success in breeding for yield and forage quality per se. Historically, yield and forage quality are objectives of high concern to farmers.

Forages are important in the world's food resources as plant materials containing relatively high amounts of structural carbohydrates which monogastrics, including man, are limited in their ability to process but which are relatively well utilized by ruminant animals (Van Soest, 1980). Feeding value of and animal response to a feedstuff are influenced by several factors including but not limited to digestibility, consumption, palatability, energy use and efficiency. Lignification during plant development has been identified as the major factor limiting extent of digestibility of cell walls and forage dry matter (Van Soest, 1982; Akin, 1989; Hartley and Ford, 1989; Jung, 1989).

Brown midrib is a visible marker associated with the reduction of lignin in corn, sorghum, and pearl millet (Kuc and Nelson, 1964; Porter et al., 1978; Cherney et al., 1988). Jung and Fahey (1983) suggested that brown midrib plants have lignin that is less polymerized and contains less phenolic monomers that can affect digestion. According to a public news release from Purdue University in 2003, the bmr gene(s) encodes caffeic acid O-methyl transferase, a lignin-producing enzyme which in conjunction with cinnamyl alcohol dehydrogenase have been shown to produce modified and reduced amounts of lignin compared to normal plants. The brown midrib trait, discovered as early as 1931, which results in reduced lignification, reduced cell-wall concentration, increased digestibility and increased voluntary intake of feed by ruminants represents the single most rapid and effective means of genetically modifying nutritional value of forage crops (Casler et al., 2003). As single-locus recessive mutations, they can be backcrossed easily into elite lines. Lignin content of brown midrib lines has been reduced by 5 to 50%; a 10 g kg⁻¹ decrease in lignin generally resulted in a 40 g kg⁻¹ increase in digestibility and increases in voluntary intake and animal performance by up to 30% (Cherney et al., 1991).

In spite of these advantages, brown midrib mutants were not used in commercial germplasm until the 1990s and widespread use of the brown midrib trait was limited by reduced yield and vigor of brown midrib phenotypes. In maize, (Zea mays L.), yield reductions associated with the brown midrib phenotype averaged ˜20% for grain, 10 to 17% for stover, and 16% for fodder (Miller et al., 1983; Lee and Brewbaker, 1984). Brown midrib lines have reduced stalk mass per unit length (Zuber et al., 1977) and increased stalk lodging (Miller et al., 1983). The effect of brown midrib loci in sorghum is generally believed to be similar to that in maize, an important impediment to commercialization (Kalton, 1988).

Brown midrib forage sorghums and sorghum× sudangrass hybrids are being introduced into the market at a very fast rate (Miller and Stroup, 2003). Some problems exist with lodging or lack of stem strength and yield drag associated with the brown midrib trait, but several hybrids combine standability, productivity, and a brown midrib phenotype.

Fritz et al. (1981) evaluated the effect of the brown midrib sorghum alleles bmr-6, bmr-12 and bmr-18 in F₂ sudangrass plants of first and second backcrosses. Despite this early work suggesting that sorghum brown midrib mutant genes can result in lower lignin percentages and higher digestibility in segregating sudangrass populations, there are no known brown midrib sudangrass varieties commercialized to date. Piper and Greenleaf sudangrass were recently compared to their brown midrib counterparts and to four highly selected brown midrib lines (30 years of breeding) in Nebraska and Wisconsin (Casler et al., 2003). While brown midrib lines averaged 9% lower in lignin and 7.2% higher in in vitro fiber digestibility than normal lines, severe forage yield reductions were observed. The brown midrib phenotype reduced forage yield by an average of 15% for first harvest and 30% for second harvest suggesting that the brown midrib phenotype was fundamentally responsible for observed limits on forage yield (Casler et al., 2003). Reduced lignification is not known to reduce regrowth per se, but there is evidence that reduced lignification can result in reduced forage yield and long-term survival of perennial forage crops (Casler et al., 2002). Severe disruptions to lignin biosynthesis can significantly reduce plant vigor and health (Jung and Ni, 1988; Casler et al., 2002).

As demonstrated by this review, there is a real need for sudangrass plants that combine productivity and disease resistance with reduced lignification, reduced cell-wall concentration, increased digestibility and increased voluntary intake of feed by ruminants. This invention provides brown midrib sudangrass plants selected for improved productivity, disease resistance, reduced lignification, reduced cell-wall concentration, and increased digestibility. The brown midrib sudangrass plants provided by this invention unexpectedly combine adaptation, productivity, and disease resistance with reduced lignification, reduced cell-wall concentration, and increased digestibility.

### SUMMARY OF THE INVENTION

This invention provides agronomically adapted brown midrib sudangrass plants and sudangrass varieties having reduced lignification when compared to adapted commercial sudangrass plants and sudangrass varieties grown under the same field conditions in North America.

The present invention provides sudangrass plants, including inbred and hybrid sudangrass plants, wherein the plants produce about a 3% or more, or about 4% or more, or about 5% or more, or about a 6% or more, or about 7% or more, or about 8% or more, or about a 9% or more, or about 10% or more, or about 11% or more, or about a 12% or more, or about 13% or more, or about 14% or more, or about a 15% or more, or about 16% or more, or about 17% or more, or about a 18% or more, or about 19% or more, or about a 20% or more, or about a 21% or more reduction in lignin concentration compared to the sudangrass variety ‘Piper’.

This invention provides sudangrass plants that have on average about 19% lower lignification compared to an adapted commercial sudangrass variety grown under the same field growing conditions in North America. This invention provides sudangrass plants that have on average about 19% lower lignification compared to an adapted commercial sudangrass variety grown under the same field growing conditions in North America, wherein the adapted commercial variety is ‘Piper’.

In another aspect the present invention provides brown midrib plants that combine reduced lignification with a desirable sudangrass phenotype. In yet another aspect the invention provides brown midrib sudangrass plants with, adaptation, productivity, and disease resistance. In still another aspect the invention provides brown midrib sudangrass plants with reduced lignification, reduced cell-wall concentration, and increased digestibility.

This invention provides brown midrib sudangrass plants, including sudangrass inbreds and hybrids, which enable on average about 20% higher weight gain per head per day for grazing beef cattle compared to an adapted commercial sudangrass variety grown under the same field growing conditions in North America. This invention provides brown midrib plants that enable on average about 20% higher weight gain per head per day for grazing beef cattle compared to an adapted commercial sudangrass variety grown under the same field growing conditions in North America, wherein the adapted commercial variety is ‘Piper’. In another aspect the present invention provides brown midrib sudangrass plants that enable on average about 20% higher weight gain per acre for grazing beef cattle compared to an adapted commercial sudangrass variety grown under the same field growing conditions in North America. This invention provides brown midrib plants that enable on average about 20% higher weight gain per acre for grazing beef cattle compared to an adapted commercial sudangrass variety grown under the same field growing conditions in North America, wherein the adapted commercial variety is ‘Piper’.

The invention also provides any of the reproductive and regenerative parts of any of the brown midrib sudangrass plants of the present invention, including but not limited to plant cells (in vivo and in vitro), cell cultures, plant parts, plant tissues and tissue cultures. Examples of such plant cells, plant tissues or plant parts include but are not limited to pollen, ovary, ovules, cotyledons, seeds, seedlings, leaflets, leaves, petioles, stems, branches, stipules, and the like.

In yet another embodiment, the present invention provides a tissue culture of regenerable cells from the brown midrib sudangrass plants of the present invention, wherein the tissue regenerates plants having all or substantially all of the morphological and physiological characteristics of the brown midrib sudangrass plants provided by the present invention. In one such embodiment, the tissue culture is derived from a plant part selected from the group consisting of leaves, roots, root tips, root hairs, anthers, pistils, stamens, pollen, ovules, flowers, seeds, embryos, stems, buds, cotyledons, hypocotyls, cells and protoplasts. In another such embodiment, the present invention includes a brown midrib sudangrass plant regenerated from the above described tissue culture.

In still another embodiment, the present invention provides any of the forage produced by brown midrib sudangrass plants of the present invention that would be consumed as fresh or preserved feed. In one such embodiment, the forage would be consumed by direct grazing. In another such embodiment, the forage would be consumed as greenchop fed to animals in confinement. In yet another such embodiment, the forage would be fed as preserved forage, also known as preserved feed. Examples of such preserved forage include but are not limited to hay, haylage, and silage.

This invention provides the cells, cell culture, tissues, tissue culture, seed, whole plants and plant parts of sudangrass inbred line designated ‘CW A.9111-1’ and having ATCC Accession No. PTA-7197.

This invention provides the cells, cell culture, tissues, tissue culture, seed, whole plants and plant parts of sudangrass inbred line designated ‘CW R.1006-55’ and having ATCC Accession No. PTA-7195.

This invention provides the cells, cell culture, tissues, tissue culture, seed, whole plants and plant parts of sudangrass hybrid designated ‘CW 1-63-21’ and having ATCC Accession No. PTA-7196, wherein this hybrid is produced by crossing inbred lines ‘CW A.9111-1’ and ‘CW R.1006-55’.

This invention also provides a cell, cell culture, tissue and/or tissue culture of regenerable cells, the cells comprising genetic material from the sudangrass inbred line named ‘CW A.9111-1’, wherein the cells regenerate plants having all or substantially all of the morphological and physiological characteristics of the sudangrass inbred line designated ‘CW A.9111-1’ and having ATCC Accession No. PTA-7197.

This invention also provides a cell, cell culture, tissue and/or tissue culture of regenerable cells, the cells comprising genetic material from the sudangrass inbred line named ‘CW R. 1006-55’, wherein the cells regenerate plants having all or substantially all of the morphological and physiological characteristics of the sudangrass inbred line designated ‘CW R.1006-55’ and having ATCC Accession No. PTA-7195.

This invention also provides a cell, cell culture, tissue and/or tissue culture of regenerable cells, the cells comprising genetic material from the sudangrass hybrid named ‘CW 1-63-21’, wherein the cells regenerate plants having all or substantially all of the morphological and physiological characteristics of the sudangrass hybrid designated ‘CW 1-63-21’, and having ATCC Accession No. PTA-7196.

This invention provides the cells, cell culture, tissues, tissue culture, seed, whole plants and plant parts of sudangrass inbred line designated ‘CW R.8904-215’ and having ATCC Accession No. PTA-7194.

This invention provides the cells, cell culture, tissues, tissue culture, seed, whole plants and plant parts of sudangrass hybrid designated ‘CW 2-43-6’ and having ATCC Accession No. PTA-7193, wherein this hybrid is produced by crossing inbred lines ‘CW A.9111-1’ and ‘CW R.8904-215’.

This invention also provides a cell, cell culture, tissue and/or tissue culture of regenerable cells, the cells comprising genetic material from the sudangrass inbred line named ‘CW R.8904-215’, wherein the cells regenerate plants having all or substantially all of the morphological and physiological characteristics of the sudangrass inbred line designated ‘CW R.8904-215’ and having ATCC Accession No. PTA-7194.

This invention also provides a cell, cell culture, tissue and/or tissue culture of regenerable cells, the cells comprising genetic material from the sudangrass hybrid named ‘CW 2-43-6’, wherein the cells regenerate plants having all or substantially all of the morphological and physiological characteristics of the sudangrass hybrid designated ‘CW 2-43-6’, and having ATCC Accession No. PTA-7193.

Using standard sudangrass breeding methods well know to one skilled in the art, the newly-developed sudangrass inbred lines (e.g., CW A.9111-1, CW R.1006-55 and CW R.8904-215) can be used to produce new sudangrass genotypes (e.g., CW 1-63-21 and CW 2-43-6) having reduced lignification, reduced cell-wall concentration, increased digestibility and other agronomic and economically beneficial traits (e.g., improved palatability, improved intake potential, and/or improved sugar content).

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

### I. Overview of the Invention

The present invention is directed to the development of agronomically adapted brown midrib sudangrass inbred lines with reduced lignification, reduced cell-wall concentration, increased digestibility and methods for identifying and isolating such plants. Furthermore, the improved sudangrass plants of the present invention are directed to the production of brown midrib sudangrass hybrids with reduced lignification, reduced cell-wall concentration, increased digestibility, adaptation, productivity, and disease resistance. In addition, the improved sudangrass plants of the present invention are directed to the production of brown midrib hybrid sudangrass hay with reduced lignification, reduced cell-wall concentration, increased digestibility, improved palatability, and increased intake potential.

### II. Definitions

As used herein, the term “sudangrass” means Sorghum bicolor ssp sudanense, formerly considered taxonomically as Sorghum sudanense. Thus, as used herein, the term “sudangrass” means any type of sudangrass typified by varieties including but not limited to Greenleaf, Piper, Sweet, Tift, and Wheeler.

As used herein, the term “sudangrass hybrid” refers to a seed, cell, whole plant, plant part (e.g., root, stem, leaf, pollen, ovule, etc.), and/or tissue culture produced from or originating from the cross of two genetically different sudangrass (Sorghum bicolor ssp. sudanense L.) parental plants, wherein one parent plant is used as the female parent plant and the other parent plant is used as the male parent plant.

As used herein, the term “brown midrib” means the phenotype produced by any of the recessive brown midrib genes (bmr₆, bmr₁₂, and bmr₁₈) when in the homozygous state.

As used herein, the term “lignification” means the deposition of lignin in plant cell walls.

As used herein, the term “callus” refers to a clump of undifferentiated plant cells that are capable of repeated cell division and growth, and in some species, can be induced to form a whole plant.

As used herein, the term “somatic tissues” refers to tissues not including germ cells or gametes. Somatic tissues are composed of vegetative tissues and cells.

As used herein, the term “somatic embryogenesis” refers to the process of embryo initiation and development from vegetative or non-gametic cells. The embryos from a given tissue source are presumed to be genetically identical.

As used herein, the term “explant” refers to a piece of tissue taken from a donor plant for culturing.

### III. Trait Determinations

Forage yield was determined by cutting forage (i.e., hay, preserved forage) in replicated trials at the late-vegetative to pre-boot growth stage, measuring fresh weight of the cut forage, drying samples with forced heated air at 55° C., determining dry matter percentage of the cut forage, and calculating forage yield on a dry matter basis. Forage Quality was determined using Near Infrared Reflectance Spectroscopy or NIRS. NIRS was conducted according to Shenk, John S. and Mark O. Westerhaus, Forage Analysis by Near Infrared Spectroscopy, In Forages Vol. II 5th ed., Ed. Robert Barnes, Darrell A Miller, C Jerry Nelson published by Iowa State University Press, Ames Iowa (1995). Weight gain per head per day was determined by intensive rotational grazing of replicated pastures with beef cattle using standard put and take stocking based on dry matter availability and calculated as an average over tester animals of the final weight minus initial weight divided by number of days grazed. Weight gain per acre was determined as the total weight gained by both tester and grazer animals. Animal performance was determined using standard methods according to Parish et al., (2003) and Hafley (1996).

The following commercial sudangrass variety is adapted for sudangrass production in North America, is the most widely used sudangrass variety, and is appropriate as a commercial check for evaluating the lignification and forage quality of newly developed sudangrass variety: ‘Piper’.

### IV. Seed Deposits

On Oct. 28, 2005, at least 2,500 seeds of each of three different sudangrass inbred lines and two sudangrass hybrids produced by crossing two of these inbred lines were deposited under the conditions of the Budapest Treaty with the American Type Culture Collection (ATCC), 10801 University Blvd., Manassas, Va. 20110-2209. The following seed deposits are representative of the instant invention:

Seed of sudangrass inbred line designated ‘CW A.9111-1’ has been given ATCC No. PTA-7197.

Seed of sudangrass inbred line designated ‘CW R. 1006-55’ has been given ATCC No. PTA-7195.

Seed of sudangrass inbred line designated ‘CW R.8904-215’ has been given ATCC No. PTA-7194.

Seed of sudangrass hybrid designated ‘CW 1-63-21’ has been given ATCC No. PTA-7196.

Seed of sudangrass hybrid designated ‘CW 2-43-6’ has been given ATCC No. PTA-7193.

### V. Cell and Tissue Culture of Sudangrass

Plants, due to their sessile nature and long life span, have developed a greater ability to endure extreme conditions and predation than have animals. Many of the processes involved in plant growth and development adapt to environmental conditions. This plasticity allows plants to alter their metabolism, growth and development to best suit their environment. Particularly important aspects of this adaptation, as far as plant tissue culture and regeneration are concerned, are the abilities to initiate cell division from almost any tissue of the plant and to regenerate lost organs or undergo different developmental pathways in response to particular stimuli. When plant cells and tissues are cultured in vitro they generally exhibit a very high degree of plasticity, which allows one type of tissue or organ to be initiated from another type. In this way, whole plants can be subsequently regenerated. This regeneration of whole organisms depends upon the concept that all plant cells can, given the correct stimuli, express the total genetic potential of the parent plant.

When cultured in vitro, all the needs, both chemical and physical, of the plant cells have to be met by the culture vessel, the growth medium and the external environment (light, temperature, etc.). The growth medium has to supply all the essential mineral ions required for growth and development. In many cases (as the biosynthetic capability of cells cultured in vitro may not replicate that of the parent plant), it must also supply additional organic supplements such as amino acids and vitamins. Many plant cell cultures, as they are not photosynthetic, also require the addition of a fixed carbon source in the form of a sugar (most often sucrose). One other vital component that must also be supplied is water, the principal biological solvent. Physical factors, such as temperature, pH, the gaseous environment, light (quality and duration) and osmotic pressure, also have to be maintained within acceptable limits.

Cultures are generally initiated from sterile pieces of a whole plant. These pieces are termed ‘explants’, and may consist of pieces of organs, such as leaves or roots, or may be specific cell types, such as pollen or endosperm. Many features of the explant are known to affect the efficiency of culture initiation. Generally, younger, more rapidly growing tissue (or tissue at an early stage of development) is most effective. Main types of cultures include but not limited to callus cultures, cell-suspension cultures, protoplasts, root cultures, shoot tip and meristem culture, embryo culture and microspore culture. Whole plants are regenerated from these cultures. Two methods of plant regeneration are widely used in plant transformation studies, i.e. somatic embryogenesis and organogenesis. In somatic (asexual) embryogenesis, embryo-like structures, which can develop into whole plants in a way analogous to zygotic embryos, are formed from somatic tissues. Organogenesis relies on the production of organs, either directly from an explant or from a callus culture.

Further reproduction of the sudangrass plants of the present invention can occur by cell and tissue culture and regeneration, wherein the tissue regenerates plants having all or substantially all of the morphological and physiological characteristics of the sudangrass plants provided by the present invention. Thus, another aspect of this invention is to provide cells which upon growth and differentiation produce sudangrass plants with reduced lignification, reduced cell-wall concentration, increased digestibility, adaptation, productivity, and disease resistance.

Yet another embodiment is a tissue culture of regenerable cells, where the cells include genetic material with reduced lignification, reduced cell-wall concentration, increased digestibility, adaptation, productivity, and disease resistance. Some embodiments include such a tissue culture that includes cultured cells derived, in whole or in part, from a plant part selected from the group consisting of leaves, roots, root tips, root hairs, anthers, pistils, stamens, pollen, ovules, flowers, seeds, embryos, stems, buds, cotyledons, hypocotyls, cells and protoplasts.

In one embodiment, this invention provides cells which upon growth and differentiation produce sudangrass plants having all or substantially all of the physiological and morphological characteristics of sudangrass inbred lines designated ‘CW A.9111-1’, ‘CW R.1006-55’ and ‘CW R.8904-215’ and sudangrass hybrids ‘CW 1-63-21’ and ‘CW 2-43-6’.

In another such embodiment, the present invention includes a sudangrass plant regenerated from the above described tissue culture.

Methods of producing sudangrass plants from tissue culture are well known by the ordinary artisan. See, for example, Jeoung et al., Hereditas 137:20-28 (2002); Zhu et al., J. Genet. Breed. 52 (2): 43-252 (1998); Zhao et al., Plant Mol. Biol. 44:789-798 (2000); Godwin and Chikwamba, Transgenic Grain Sorghum (Sorghum bicolor) plants via Agrobacterium, In Improvement of Cereal Quality by Genetic Engineering, Henry and Ronalds (ed.), Plenum Press (1994); Cassas et al., In Vitro Cell Dev, Biol. Plant 33:92-100 (1997); Able et al., In Vitro Cell Dev, Biol. Plant 37:341-348 (2001); Grootboom and O'Kennedy, Genetic Enhancement of Nutritional Quality of Grain Sorghum, In AFRIPRO 2003 Conference—Workshop on the proteins of Sorghum and Millet: Enhancing Nutritional and Functional Properties for Africa, Belton and Taylor (ed.); and U.S. Pat. No. 6,369,298, each of which is incorporated herein in their entirety.

Initiation of callus from immature zygotic embryos of sudangrass inbred lines designated ‘CW A.9111-1’, ‘CW R.1006-55’ and ‘CW R.8904-215’ and the sudangrass hybrids ‘CW 1-63-21’ and ‘CW 2-43-6’ can be achieved on I₆ medium supplemented with 2,4-dichlorophenoxyacetic acid (2,4-D). See, for example, Jeoung et al., Hereditas 137:20-28 (2002). Whole sudangrass plants can be produced from the callus tissue, wherein the sudangrass plants have the same or substantially the same morphological and physiological characteristics as the plant from which the calli were derived.

### VI. Uses of Sudangrass

The many uses of sudangrass are discussed throughout the specification; some of these uses are further discussed in this section.

The sudangrass inbreds and hybrids of the present invention can be planted and grown under any conditions conducive to sudangrass germination and growth. The sudangrass inbreds and hybrids of this invention can be utilized for any purpose for which sudangrass is known to be used, including but not limited to using it as a ground cover, to produce sudangrass inbred and hybrid seed, and as a feed for both wild and domesticated animals, such as birds and mammals.

The sudangrass inbred and hybrid seed of the present invention can be planted under any conditions that result in germination of sudangrass seed and growth of sudangrass plants. Alternatively, the seeds may be germinated in one location and the resulting seedlings/plants can be transplanted to another location. Regardless of whether they result from direct seeding or transplantation, the sudangrass plants can be harvested at any growth stage or allowed to mature so as to produce seed. The resulting seed can be harvested with/without other plant parts depending on the ultimate use of the seed as a seed crop or as feed.

In another embodiment, the invention also includes using the sudangrass plants of the present invention in methods of producing animal feeds and in methods of administering such feeds to animals.

In a further aspect, the invention contemplates feed for ruminants comprising the sudangrass plants provided by the present invention. Sudangrass is basic forage for maximizing ruminant animal production and provides an important source of nutrients for ruminant livestock such as dairy and beef cattle and sheep.

Feed which includes sudangrass plants of the present invention can take many forms including but not limited to direct pasturage, greenchop, silage, hay, and haylage. Direct pasturage can be done using pure stands of sudangrass or mixtures of sudangrass with other types of plants. Greenchop is harvested and fed to the animals shortly thereafter. The silage, hay and haylage can be harvested and stored in many different ways well known by those skilled in the art. Examples of such harvesting and storage methods include but are not limited to wrapped bales, (e.g., using plastic wrapping), unwrapped bales, pile silos, tower silos, bunker silos, etc. The bales can take many different sizes (e.g., small, medium or large bales) and many different shapes (e.g., round bales, square bales, etc.).

Sudangrass can be fed directly to animals or mixed with various other different types of feeds (e.g., soybean, corn, sorghum, etc.) and/or various types of feed supplements (e.g., amino acids, protein supplements, vitamins, salts, minerals, liquid feeds, medicines, etc.) prior or during feeding so as to provide a more balanced ration, sometimes referred to as a “Total Mixed Ration”.

The foregoing detailed description has been given for clearness of understanding only and no unnecessary limitations should be understood therefrom as modifications will be obvious to those skilled in the art.

### EXAMPLES

### Example 1

CW 1-63-21 is a single cross hybrid involving two advanced generation inbred lines, CW A.9111-1 and CW R.1006-55. The female parent CW A.9111-1 has cytoplasmic male sterility derived from Sorghum bicolor, cv ‘Dwarf Yellow Milo’. The female parent CW A.9111-1 and its male fertile nonrestorer (maintainer) counterpart CW B. 9111-1 were simultaneously developed by pedigree selection from proprietary Cal/West Seeds sudangrass germplasm pools with diverse genetic background including derivatives of Piper, Greenleaf, and Sweet. The nonrestorer character was incorporated from S. bicolor, cv ‘Redlan’.

The pollinator inbred line CW R. 1006-55 was developed by inbreeding and pedigree selection within a proprietary Cal/West Seeds sudangrass population developed by recurrent selection. The bmr-₁₂ gene integrated into CW A.9111-1, CW B.9111-1, and CW R. 1006-55 was incorporated from Purdue University sorghum mutant bmr-12 (Porter et al., 1978). Seed was propagated by panicle-to-row selection until the F₁₀ generation, at which time it was bulk increased for commercial production.

### Example 2

Inferior forage yield potential and vegetative productivity has been associated with expression of the bmr gene in brown midrib sudangrass (Calser et al., 2003). Development of improved inbred lines and capture of heterosis in specific hybrid combinations has been the basis for the hybrid seed industry. CW 1-63-21 brown midrib sudangrass was identified from a group of 20 hybrids produced in 2001 from recombination among a group of recently developed inbred lines. CW 1-63-21 brown midrib sudangrass has forage yield equal to or higher than adapted check sudangrass varieties grown at the same time in the same location.

TABLE 2
Yield performance of CW 1-63-21 brown midrib sudangrass compared to adapted check varieties
‘Piper’, ‘Greeleaf’, and ‘Sweet’ grown at the same time in the same locations.

| | | | Yield of | Yield of | Yield of | Yield of | Mean Yield |
|---|---|---|---|---|---|---|---|
| | Date | No. of | CW 1-63-21 | Piper | Greenleaf | Sweet | of Trial |
| Location | Seeded | Harvests | (Tons/acre) | (Tons/acre) | (Tons/acre) | (Tons/acre) | (Tons/acre) |
| Woodland, CA | May 26, 2002 | 3 | 9.59 | 9.68 | 9.54 | 9.64 | 8.80 |
| West Salem, WI | May 29, 2002 | 3 | 6.71 | 6.41 | 6.33 | 5.86 | 6.14 |
| Woodland, CA | May 19, 2003 | 3 | 14.21 | 12.41 | 12.44 | 10.47 | 12.20 |
| West Salem, WI | May 28, 2003 | 3 | 7.97 | 8.02 | 7.87 | 7.54 | 7.07 |
| Woodland, CA | May 10, 2004 | 4 | 13.02 | 11.88 | 10.17 | 11.39 | 11.58 |
| West Salem, WI | May 23, 2004 | 2 | 3.95 | 4.90 | 4.67 | 4.35 | 3.87 |
| TOTAL | | 18 | 55.45 | 53.3 | 51.02 | 49.25 | 49.66 |
| Average | | | 9.24 | 8.88 | 8.50 | 8.21 | 8.28 |

### Example 3

Cell Wall Concentration (CWC) is the limiting factor in determining forage intake by ruminant animals (Van Soest, 1980). Neutral Detergent Fiber (NDF) estimates the CWC of forages. Among parameters commonly used to estimate forage quality, NDF is most highly correlated with and is the best predictor of forage intake potential. CW 1-63-21 brown midrib sudangrass has lower CWC as measured by NDF compared to the adapted check sudangrass variety when grown at the same time in the same locations.

TABLE 3
Neutral Detergent Fiber (NDF) of CW 1-63-21 brown midrib sudangrass compared to
adapted check variety ‘Piper’ grown at the same time in the same location.

| | | | | | Expression | |
|---|---|---|---|---|---|---|
| | | | NDF of | | of CW 1-63-21 | Mean NDF |
| | Date | No. of | CW 1-63-21 | NDF of | Relative to | of Trial |
| Location | Seeded | Harvests | (%) | Piper (%) | Piper | (Tons/acre) |
| Woodland, CA | May 26, 2002 | 3 | 50.11 | 51.99 | −3.6% | 48.97 |
| West Salem, WI | May 29, 2002 | 2 | 54.62 | 55.60 | −1.8% | 54.60 |
| Woodland, CA | May 19, 2003 | 3 | 57.58 | 59.90 | −3.9% | 57.81 |
| West Salem, WI | May 28, 2003 | 2 | 61.17 | 64.10 | −4.6% | 60.85 |
| Woodland, CA | May 10, 2004 | 4 | 58.60 | 61.16 | −4.2% | 60.65 |
| West Salem, WI | May 23, 2004 | 2 | 59.00 | 62.12 | −5.0% | 60.04 |
| TOTAL | | 16 | 341.08 | 354.87 | | 342.92 |
| Average | | | 56.85 | 59.14 | −3.9% | 57.15 |

### Example 4

Lignin is a phenolic polymer that is cross-linked to cellulose and hemicellulose polysaccharides in plant cell walls. Lignin is non digestible and reduces the digestibility of the partially digestible hemicellulose fraction of plant cell walls by physically restricting cell wall exposure to rumen microorganisms. CW 1-63-21 brown midrib sudangrass has lower ADL compared to the adapted check sudangrass variety when grown at the same time in the same locations.

TABLE 4
Acid Detergent Lignin (ADL) of CW 1-63-21 brown midrib sudangrass compared to adapted
check sudangrass variety ‘Piper’ grown at the same time in the same location.

| | | | | | Expression | |
|---|---|---|---|---|---|---|
| | | | ADL of | | of CW 1-63-21 | Mean ADL |
| | Date | No. of | CW 1-63-21 | ADL of | Relative to | of Trial |
| Location | Seeded | Harvests | (%) | Piper (%) | Piper | (%) |
| Woodland, CA | May 26, 2002 | 3 | 7.54 | 8.76 | −13.9% | 7.72 |
| West Salem, WI | May 29, 2002 | 2 | 7.68 | 9.30 | −17.4% | 8.82 |
| Woodland, CA | May 19, 2003 | 3 | 5.92 | 7.54 | −21.5% | 6.41 |
| West Salem, WI | May 28, 2003 | 2 | 5.61 | 7.19 | −22.0% | 6.06 |
| Woodland, CA | May 10, 2004 | 4 | 5.73 | 7.67 | −25.3% | 5.63 |
| West Salem, WI | May 23, 2004 | 2 | 6.23 | 7.52 | −17.2% | 6.58 |
| TOTAL | | 16 | 38.71 | 47.98 | | 41.22 |
| Average | | | 6.45 | 8.00 | −19.3% | 6.87 |

### Example 5

Cell wall digestibility has been shown to be a major factor in explaining why feeds with similar protein and cell wall concentrations result in significantly different animal performance as measured by milk or meat output. Digestibility of the NDF fraction, Neutral Detergent Fiber Digestibility (NDFD), is considered an accurate measure of cell wall digestibility. CW 1-63-21 brown midrib sudangrass has higher NDFD compared to the adapted check sudangrass variety when grown at the same time in the same location.

TABLE 5
Neutral Detergent Fiber Digestibility (NDFD) of CW 1-63-21 sudangrass compared to
adapted check variety ‘Piper’ grown at the same time in the same location.

| | | | | | Expression | |
|---|---|---|---|---|---|---|
| | | | NDFD of | | of CW 1-63-21 | Mean NDFD |
| | Date | No. of | CW 1-63-21 | NDFD of | Relative to | of Trial |
| Location | Seeded | Harvests | (%) | Piper (%) | Piper | (Tons/acre) |
| Woodland, CA | May 26, 2002 | 3 | 74.15 | 70.83 | 4.7% | 76.47 |
| West Salem, WI | May 29, 2002 | 2 | 77.62 | 72.69 | 6.8% | 75.67 |
| Woodland, CA | May 19, 2003 | 3 | 81.37 | 74.39 | 9.4% | 79.82 |
| West Salem, WI | May 28, 2003 | 2 | 77.64 | 71.90 | 8.0% | 75.97 |
| Woodland, CA | May 10, 2004 | 4 | 80.59 | 72.76 | 10.8% | 79.72 |
| West Salem, WI | May 23, 2004 | 2 | 70.71 | 67.07 | 5.4% | 69.91 |
| TOTAL | | 16 | 462.08 | 429.64 | | 457.56 |
| Average | | | 77.01 | 71.61 | 7.6% | 76.26 |

### Example 6

Crude protein (CP) of hay has been shown to be an excellent predictor of digestible protein. Protein in the ruminant ration is necessary for providing essential amino acids required by the animal for health and productivity. CW 1-63-21 brown midrib sudangrass has higher CP compared to the adapted check sudangrass variety when grown at the same time in the same locations.

TABLE 6
Crude Protein (CP) of CW 1-63-21 sudangrass compared to adapted check
variety ‘Piper’ grown at the same time in the same location.

| | | | | | Expression | |
|---|---|---|---|---|---|---|
| | | | CP of | | of CW 1-63-21 | Mean CP |
| | Date | No. of | CW 1-63-21 | CP of | Relative to | of Trial |
| Location | Seeded | Harvests | (%) | Piper (%) | Piper | (Tons/acre) |
| Woodland, CA | May 26, 2002 | 3 | 15.47 | 15.77 | −1.9% | 16.14 |
| West Salem, WI | May 29, 2002 | 2 | 14.47 | 13.87 | 4.3% | 14.27 |
| Woodland, CA | May 19, 2003 | 3 | 17.41 | 16.20 | 7.5% | 17.09 |
| West Salem, WI | May 28, 2003 | 2 | 15.95 | 14.80 | 7.8% | 15.76 |
| Woodland, CA | May 10, 2004 | 4 | 16.96 | 15.78 | 7.5% | 17.55 |
| West Salem, WI | May 23, 2004 | 2 | 17.20 | 15.96 | 7.8% | 16.60 |
| TOTAL | | 16 | 97.46 | 92.38 | | 97.41 |
| Average | | | 16.24 | 15.40 | 5.5% | 16.24 |

### Example 7

Differential productivity of grazing ruminant animals has been shown to result from differences in cell wall concentration, lignification, cell wall digestibility, and protein content. CW 1-63-21 brown midrib sudangrass enables higher weight gain per head per day and weight gain per acre for beef cattle (Bos taurus) compared to the adapted check sudangrass variety when grown at the same time in the same location

TABLE 7.a
Weight gain (pounds) per head per day for beef cattle grazing CW 1-63-21 sudangrass compared
to adapted check variety ‘Piper’ grown at the same time in the same location.

| | | | | | Expression |
|---|---|---|---|---|---|
| | | No. of | Weight Gain | Weight Gain | of CW 1-63-21 |
| | Date | Grazing | CW 1-63-21 | Piper | Relative to |
| Location | Seeded | Cycles | (pounds/head/day) | (pounds/head/day) | Piper |
| Prairie, MS | Jun. 01, 2005 | 5 | 1.8 | 1.5 | 20.0% |

Replicated 5-acre pastures managed with intensive rotational grazing for 56 days and a stocking rate of 2-8 head per acre based on dry matter availability.

TABLE 7.b
Weight gain (pounds) per acre for beef cattle grazing CW 1-63-21 sudangrass compared
to adapted check variety ‘Piper’ grown at the same time in the same location.

| | | | | | Expression |
|---|---|---|---|---|---|
| | | No. of | Weight Gain | Weight Gain | of CW 1-63-21 |
| | Date | Grazing | CW 1-63-21 | Piper | Relative to |
| Location | Seeded | Cycles | (pounds/acre) | (pounds/acre) | Piper |
| Prairie, MS | Jun. 01, 2005 | 5 | 130.4 | 108.8 | 19.9% |

Replicated 5-acre pastures managed with intensive rotational grazing for 56 days and a stocking rate of 2-8 head per acre based on dry matter availability.

### Example 8

CW 2-43-6 is a single cross hybrid involving two advanced generation inbred lines, CW A.9111-1 and CW R.8904-215. The female parent CW A.9111-1 has cytoplasmic male sterility derived from Sorghum bicolor, cv ‘Dwarf Yellow Milo’. The female parent CW A.9111-1 and its male fertile nonrestorer (maintainer) counterpart CW B.9111-1 were simultaneously developed by pedigree selection from proprietary Cal/West Seeds sudangrass germplasm pools with diverse genetic background including derivatives of Piper, Greenleaf, and Sweet. The nonrestorer character was incorporated from S. bicolor, cv ‘Redlan’.

The pollinator inbred line CW R.8904-215 was developed by inbreeding and pedigree selection within a proprietary Cal/West Seeds sudangrass population developed by recurrent selection. The bmr₁₂ gene integrated into CW A.9111-1, CW B.9111-1, and CW R.8904-215 was incorporated from Purdue University sorghum mutant bmr-12 (Porter et al., 1978). Seed was propagated by panicle-to-row selection until the F₁₀ generation, at which time it was bulk increased for commercial production.

The brown midrib sudangrass hybrid CW 2-43-6 has forage yield equal to or higher than the adapted sudangrass check varieties grown at the same time in the same locations. The brown midrib hybrid CW 2-43-6 has lower NDF and ADL and higher NDFD and CP than the adapted check sudangrass variety when grown at the same time in the same locations.

TABLE 8.a
Yield performance of CW 2-43-6 brown midrib sudangrass compared to adapted check varieties ‘Piper’,
‘Greeleaf’, and ‘Sweet’ grown at the same time in the same locations.

| | | | Yield of | Yield of | Yield of | Yield of | Mean Yield |
|---|---|---|---|---|---|---|---|
| | Date | No. of | CW 2-43-6 | Piper | Greenleaf | Sweet | of Trial |
| Location | Seeded | Harvests | (Tons/acre) | (Tons/acre) | (Tons/acre) | (Tons/acre) | (Tons/acre) |
| Woodland, CA | May 19, 2003 | 3 | 14.79 | 12.41 | 12.44 | 10.47 | 12.20 |
| West Salem, WI | May 28, 2003 | 3 | 7.76 | 8.02 | 7.87 | 7.54 | 7.07 |
| Woodland, CA | May 10, 2004 | 4 | 12.71 | 11.88 | 10.17 | 11.39 | 11.58 |
| West Salem, WI | May 23, 2004 | 2 | 4.45 | 4.90 | 4.67 | 4.35 | 3.87 |
| TOTAL | | 12 | 39.71 | 37.21 | 35.15 | 33.75 | 34.72 |
| Average | | | 9.93 | 9.30 | 8.79 | 8.44 | 8.68 |

TABLE 8.b
Neutral Detergent Fiber (NDF) of CW 2-43-6 brown midrib sudangrass compared to adapted
check variety ‘Piper’ grown at the same time in the same location.

| | | | | | Expression | |
|---|---|---|---|---|---|---|
| | | | NDF of | | of CW 2-43-6 | Mean NDF |
| | Date | No. of | CW 2-43-6 | NDF of | Relative to | of Trial |
| Location | Seeded | Harvests | (%) | Piper (%) | Piper | (Tons/acre) |
| Woodland, CA | May 19, 2003 | 3 | 57.58 | 59.90 | −3.0% | 57.81 |
| West Salem, WI | May 28, 2003 | 2 | 61.17 | 64.10 | −3.5% | 60.85 |
| Woodland, CA | May 10, 2004 | 4 | 58.60 | 61.16 | −4.5% | 60.65 |
| West Salem, WI | May 23, 2004 | 2 | 59.00 | 62.12 | −2.2% | 60.04 |
| TOTAL | | 11 | 239.07 | 247.28 | | 239.35 |
| Average | | | 59.77 | 61.82 | −3.3% | 59.84 |

TABLE 8.c
Acid Detergent Lignin (ADL) of CW 2-43-6 brown midrib sudangrass compared to adapted
check sudangrass variety ‘Piper’ grown at the same time in the same location.

| | | | | | Expression | |
|---|---|---|---|---|---|---|
| | | | ADL of | | of CW 2-43-6 | Mean ADL |
| | Date | No. of | CW 2-43-6 | ADL of | Relative to | of Trial |
| Location | Seeded | Harvests | (%) | Piper (%) | Piper | (%) |
| Woodland, CA | May 19, 2003 | 3 | 6.14 | 7.54 | −18.6% | 6.41 |
| West Salem, WI | May 28, 2003 | 2 | 6.14 | 7.19 | −14.6% | 6.06 |
| Woodland, CA | May 10, 2004 | 4 | 5.84 | 7.67 | −23.9% | 5.63 |
| West Salem, WI | May 23, 2004 | 2 | 6.89 | 7.52 | −8.4% | 6.58 |
| TOTAL | | 11 | 25.01 | 29.92 | | 24.68 |
| Average | | | 6.25 | 7.48 | −16.4% | 6.17 |

TABLE 8.d
Neutral Detergent Fiber Digestibility (NDFD) of CW 2-43-6 sudangrass compared to
adapted check variety ‘Piper’ grown at the same time in the same location

| | | | | | Expression | |
|---|---|---|---|---|---|---|
| | | | NDFD of | | of CW 2-43-6 | Mean NDFD |
| | Date | No. of | CW 2-43-6 | NDFD of | Relative to | of Trial |
| Location | Seeded | Harvests | (%) | Piper (%) | Piper | (Tons/acre) |
| Woodland, CA | May 19, 2003 | 3 | 80.69 | 74.39 | 8.5% | 79.82 |
| West Salem, WI | May 28, 2003 | 2 | 75.19 | 71.90 | 4.6% | 75.97 |
| Woodland, CA | May 10, 2004 | 4 | 80.10 | 72.76 | 10.1% | 79.72 |
| West Salem, WI | May 23, 2004 | 2 | 68.54 | 67.07 | 2.2% | 69.91 |
| TOTAL | | 11 | 304.52 | 286.12 | | 305.42 |
| Average | | | 76.13 | 71.53 | 6.4% | 76.36 |

TABLE 8.e
Crude Protein (CP) of CW 2-43-6 sudangrass compared to adapted check
variety ‘Piper’ grown at the same time in the same location.

| | | | | | Expression | |
|---|---|---|---|---|---|---|
| | | | CP of | | of CW 2-43-6 | Mean CP |
| | Date | No. of | CW 2-43-6 | CP of | Relative to | of Trial |
| Location | Seeded | Harvests | (%) | Piper (%) | Piper | (Tons/acre) |
| Woodland, CA | May 19, 2003 | 3 | 16.80 | 16.20 | 3.7% | 17.09 |
| West Salem, WI | May 28, 2003 | 2 | 15.80 | 14.80 | 6.8% | 15.76 |
| Woodland, CA | May 10, 2004 | 4 | 16.89 | 15.78 | 7.0% | 17.55 |
| West Salem, WI | May 23, 2004 | 2 | 16.57 | 15.96 | 3.8% | 16.60 |
| TOTAL | | 11 | 66.06 | 62.74 | | 67.00 |
| Average | | | 16.52 | 15.69 | 5.3% | 16.75 |

### Example 9

CW 3-47-10 is a single cross hybrid involving two advanced generation inbred lines, CW A.9111-2 and CW R.1006-56. The female parent CW A.9111-2 has cytoplasmic male sterility derived from Sorghum bicolor, cv ‘Dwarf Yellow Milo’. The female parent CW A.9111-2 and its male fertile nonrestorer (maintainer) counterpart CW B.9111-2 were simultaneously developed by pedigree selection from proprietary Cal/West Seeds sudangrass germplasm pools with diverse genetic background including derivatives of Piper, Greenleaf, and Sweet. The nonrestorer character was incorporated from S. bicolor, cv ‘Redlan’.

The pollinator inbred line CW R. 1006-56 was developed by inbreeding and pedigree selection within a proprietary Cal/West Seeds sudangrass population developed by recurrent selection. The bmr₁₂ gene integrated into CW A.9111-2, CW B.9111-2, and CW R. 1006-56 was incorporated from Purdue University sorghum mutant bmr-12 (Porter et al., 1978). Seed was propagated by panicle-to-row selection until the F₁₀ generation, at which time it was bulk increased for commercial production.

The brown midrib sudangrass hybrid CW 3-47-10 has forage yield equal to or higher than the adapted sudangrass check varieties grown at the same time and in the same locations. The brown midrib hybrid CW 3-47-10 has lower NDF and ADL and higher NDFD and CP than the adapted check sudangrass variety when grown at the same time in the same locations.

TABLE 9.a
Yield performance of CW 3-47-10 brown midrib sudangrass compared to adapted check varieties
‘Piper’, ‘Greeleaf’, and ‘Sweet’ grown at the same time in the same locations.

| | | | Yield of | Yield of | Yield of | Yield of | Mean Yield |
|---|---|---|---|---|---|---|---|
| | Date | No. of | CW 3-47-10 | Piper | Greenleaf | Sweet | of Trial |
| Location | Seeded | Harvests | (Tons/acre) | (Tons/acre) | (Tons/acre) | (Tons/acre) | (Tons/acre) |
| Woodland, CA | May 10, 2004 | 4 | 12.71 | 11.88 | 10.17 | 11.39 | 11.58 |
| West Salem, WI | May 23, 2004 | 2 | 4.45 | 4.90 | 4.67 | 4.35 | 3.87 |
| Woodland, CA | May 25, 2005 | 3 | 13.96 | 12.62 | 12.27 | 11.20 | 10.85 |
| TOTAL | | 9 | 31.12 | 29.40 | 27.11 | 26.94 | 26.30 |
| Average | | | 7.78 | 7.35 | 6.78 | 6.74 | 6.58 |

TABLE 9.b
Neutral Detergent Fiber (NDF) of CW 3-47-10 brown midrib sudangrass compared to
adapted check variety ‘Piper’ grown at the same time in the same location.

| | | | | | Expression | |
|---|---|---|---|---|---|---|
| | | | NDF of | | of CW 3-47-10 | Mean NDF |
| | Date | No. of | CW 3-47-10 | NDF of | Relative to | of Trial |
| Location | Seeded | Harvests | (%) | Piper (%) | Piper | (Tons/acre) |
| Woodland, CA | May 10, 2004 | 4 | 56.55 | 61.16 | −7.5% | 60.65 |
| West Salem, WI | May 23, 2004 | 2 | 57.75 | 62.12 | −7.0% | 60.04 |
| TOTAL | | 6 | 114.30 | 123.28 | | 120.69 |
| Average | | | 57.15 | 61.64 | −7.3% | 60.35 |

TABLE 9.c
Acid Detergent Lignin (ADL) of CW 3-47-10 brown midrib sudangrass compared to adapted
check sudangrass variety ‘Piper’ grown at the same time in the same location.

| | | | | | Expression | |
|---|---|---|---|---|---|---|
| | | | ADL of | | of CW 3-47-10 | Mean ADL |
| | Date | No. of | CW 3-47-10 | ADL of | Relative to | of Trial |
| Location | Seeded | Harvests | (%) | Piper (%) | Piper | (%) |
| Woodland, CA | May 10, 2004 | 4 | 5.66 | 7.67 | −26.2% | 5.63 |
| West Salem, WI | May 23, 2004 | 2 | 6.29 | 7.52 | −16.4% | 6.58 |
| TOTAL | | 6 | 11.95 | 15.19 | | 12.21 |
| Average | | | 5.98 | 7.60 | −21.3% | 6.11 |

TABLE 9.d
Neutral Detergent Fiber Digestibility (NDFD) of CW 3-47-10 sudangrass compared to
adapted check variety ‘Piper’ grown at the same time in the same location.

| | | | | | Expression | |
|---|---|---|---|---|---|---|
| | | | NDFD of | | of CW 3-47-10 | Mean NDFD |
| | Date | No. of | CW 3-47-10 | NDFD of | Relative to | of Trial |
| Location | Seeded | Harvests | (%) | Piper (%) | Piper | (Tons/acre) |
| Woodland, CA | May 10, 2004 | 4 | 81.87 | 72.76 | 12.5% | 79.72 |
| West Salem, WI | May 23, 2004 | 2 | 70.92 | 67.07 | 5.7% | 69.91 |
| TOTAL | | 6 | 152.79 | 139.83 | | 149.63 |
| Average | | | 76.40 | 69.92 | 9.3% | 74.82 |

TABLE 9.e
Crude Protein (CP) of CW 3-47-10 sudangrass compared to adapted check
variety ‘Piper’ grown at the same time in the same location.

| | | | | | Expression | |
|---|---|---|---|---|---|---|
| | | | CP of | | of CW 3-47-10 | Mean CP |
| | Date | No. of | CW 3-47-10 | CP of | Relative to | of Trial |
| Location | Seeded | Harvests | (%) | Piper (%) | Piper | (Tons/acre) |
| Woodland, CA | May 10, 2004 | 4 | 17.53 | 15.78 | 11.1% | 17.55 |
| West Salem, WI | May 23, 2004 | 2 | 16.59 | 15.96 | 3.9% | 16.60 |
| TOTAL | | 6 | 34.12 | 31.74 | | 34.15 |
| Average | | | 17.06 | 15.87 | 7.5% | 17.08 |

### SELECTED REFERENCES

Able, J. R., Rathus, C., and Godwin, I. D. 2001. The investigation of optimal bombardment parameters for transient and stable transgene expression in sorghum. In Vitro Cell Dev, Biol. Plant 37:341-348.
Akin, D. E. 1989. Histological and physical factors affecting digestibility of forages. Agron. J. 81:17-25.
Anderson, B. A. and Guyer, P. 1986. Summer Annual Forage Grasses. University of Nebraska Lincoln, NebGuide G74-171-A
Armah-Agyeman, R., Loiland, J., Karow, R., and Bean, B. 2002. Sudangrass. Dryland cropping systems. Oregon State University Extension Service, EM-8793.
Casler, M. D, Buxton, D. R., and Vogel, K. P. 2002. Genetic modification of lignin concentration affects fitness of perennial herbaceous plants. Theor. Appl. Genet. 104:127-131.
Casler, M. D., Pedersen, J. F., and Undersander, D. J. 2003. Forage yield and economic losses associated with the brown-midrib trait in sudangrass. Crop Sci. 43:782-789.
Cassas, A. M., Kononowicz, A. K., Hann, T. G., Zhang, L. Tomes, D. T., Bressan, R. A., and Hasegawa, P. M. 1997, Transgenic sorghum plants obtained after microprojectile bombardment of immature inflorescence. In Vitro Cell Dev, Biol. Plant 33:92-100.
Celarier, R. P. 1958. Cytotaxonomy of the Andropogoneae. III. Subtribe Sorgheae, genus Sorghum. Cytologia 23:395-418.
Cherney, J. H., Axtel, J. D., Hassen, M. M., and Anliker, K. S. 1988. Forage quality characterization of a chemically induced bmr mutant of pearl millet. Crop Sci. 28:783-787.
Cherney, J. H., Cherney, D. J. R., Akin, D. E., and Axtell, J. D. 1991. Potential of brown-midrib, low-lignin mutants for improving forage quality. Adv. Agron. 46:157-198.
Davis, J. R., Huisman, O. C., Westerman, D. C., Everson, D. O., et al. 2004. Some Unique Benefits With Sudangrass For Improved U.S. #1 Yields and Size of Russet Burbank Potatoes. American Journal of Potato Research 81: 403-413.
Fritz, J. O., R. P. Cantrell, V. L. Lochtenberg, J. D. Axtell and J. M. Hertel. 1981. Brown Midrib Mutants in Sudangrass and Grain Sorghum. Crop Science 21:706-709.
Godwin, I. D. and Chikwamba, R. 1994. Transgenic Grain Sorghum (Sorghum bicolor) plants via Agrobacterium, In Improvement of Cereal Quality by Genetic Engineering, Henry, R. J. and Ronalds, J. A. (ed.), Plenum Press.
Grootboom, A. and O'Kennedy, M. M. 2003. Genetic Enhancement of Nutritional Quality of Grain Sorghum, In AFRIPRO 2003 Conference—Workshop on the proteins of Sorghum and Millet: Enhancing Nutritional and Functional Properties for Africa, Belton, P. S. and Taylor, J. R. N. (ed.).
Hafley, J. L. 1996. Comparison of Marshall and Surrey Ryegrass for Continuous and Rotational Grazing. J. Animal Sci. 74:2269-2275.
Harlan, J. R. and de Wet, J. M. J. 1972. A simplified classification of cultivated sorghum. Crop Sci., 12: 172-176.
Hartley, R. D. and Ford, C. W. 1989. Phenolic constituents of plant cell walls and wall degradability. P. 137-145. In Lewis, N. G. and Paice, M. G. (ed.) Plant cell wall polymers: Biogenesis and biodegradation. ACS Symp. Ser. 399. Am. Chem. Soc., Washington, D.C.
Jeoung, J. M., Krishnaveni, S., Muthukrishnan, S., Trick, H. N., and Liang, G. H. 2002. Optimization of sorghum transformation parameters using genes for green fluorescent protein and B-glucuronidase as visual markers. Hereditas 137:20-28.
Jung, H. G. 1989. Forage lignins and their effects on fiber digestibility. Agron. J. 81:33-38.
Jung, H. G. and Fahey, G. C. 1983. Nutritional implications of phenolic monomers and lignin: a review. J. Anim. Sci 57:206-219.
Jung, H. G. and Ni, W. 1988. Lignification of plant cell walls: Impact of genetic manipulation. Proc. Natl. acad. Sci. USA 95:12742-12743.
Kalton, R. R. 1988. Overview of the forage sorghums. p. 1-12 In D. Wilkerson (ed.) Proc. 43^{rd} Corn Sorghum Res. Conf. 8-9 Dec., 1988, Chicago, Ill. Am. Seed Trade Assoc., Washington, D.C.
Kuc, J. and Nelson, O. E. 1964 The abnormal lignins produced by the brown midrib mutants of maize. I. The brown midrib-1 mutants. Arch. Biochem. Biophys. 105:103-113.
Lee, M. H and Brewbaker, J. L. 1984. Effect of brown midrib-3 on yields and yield components of maize. Crop Sci. 24:105-108.
Miller, F. R. and Stroup, J. A. 2003. Brown midrib forage sorghum, sudangrass, and corn: What is the potential? Proc. 33^{rd} California Alfalfa and Forage Symposium. pp. 143-151.
Miller, J. E., Geadelman, J. L., and Marten, G. C. 1983. Effect of the brown midrib-allele on maize silage quality and yield. Crop Sci. 23:493-496.
Mishanec, J. J. 1996. Onion Growers See Beneficial Effects of Sudangrass. Cornell University. New York State Integrated Pest Management Program Annual Report 1997. p. 29.
Parish, J. A., McCann, M. A., Watson, R. H., Paiva, N. N., Hoveland, C. S., Parks, A. H., Upchurch, B. L., Hill, N. S., and Bouton, J. H. 2003. Use of Non-Ergot Alkaloid Producing Endophytes for Alleviating Tall Fescue Toxicosis In Stocker Cattle. J. Animal Sci. 81:2856-2868.
Porter, K. S., Axtell, J. D., Lechtenberg, V. L., and Colenbrander, V. F. 1978. Phenotype, fiber composition, and in vitro dry matter disappearance of chemically induced brown midrib mutants of sorghum. Crop Sci. 18:205-208
Shenk, J. S. and Westerhaus, M. O. 1995. Forage Analysis by Near Infrared Spectroscopy, In Forages Vol. II 5th ed., Ed. Robert Barnes, Darrell A Miller, C Jerry Nelson published by Iowa State University Press, Ames Iowa.
Shertz, K. F. and Dalton, L. G. 1980. Sorghum 41:577-588, In Hybridization of Crop Plants, Fehr et al. (ed.), American Society of Agronomy Inc.
Undersander, D. J. April 2003. Sorghums, Sudangrasses, and Sorghum× Sudangrass Hybrids. University of Wisconsin Cooperative Extension Service. Focus on Forage, Vol. 5: No. 5.
Van Soest, P. J. 1980. Composition and Nutritive Value of Forages 6:53-63, In Forages The Science of Grassland Agriculture, Heath et al. (ed), The Iowa State University Press, Ames, Iowa.
Van Soest, P. J. 1982. Nutritional ecology of the ruminant. O & B Books, Corvallis, Oreg.
Viaene N M, and GS Abawi. 1998. Management of Meloidogyne hapla on lettuce in organic soil with sudangrass as a cover crop. Plant Dis 82:945-952.
Zhao, Z. Y., Cai, T., Tagliani, L., Miller, M., Wang, N., Pang, H., Rudert, R., Schroder, S., Hondred, D., Smetzler, J., and Pierce, D. 2000. Agrobacterium-mediated sorghum transformation. Plant Mol. Biol. 44:789-798.
Zhu, H., Muthukrishnan, S., Krishnaveni, S., Jeoung, J. M., and Liang, G.H.1998. Biolistic transformation of sorghum using a rice chitinase gene. J. Genet. Breed. 52 (2): 43-252.
Zuber, M. S., Colbert, T. R., and Bauman, L. F. 1977. Effect of brown midrib-3 in maize (Zea mays L.) on stalk strength. Z. Pflanzenuchtg. 79:310-314.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures of each and every patent, patent application, and publication cited herein including but limited to the references listed immediately above under ‘Selected References’ are hereby incorporated herein by reference in their entirety.

1-21. (canceled)

22. A sudangrass hybrid plant designated ‘CW 2-43-6’, representative seed of said hybrid having been deposited under ATCC Accession No. PTA-7193.

23. A seed which produces the sudangrass hybrid plant of claim 22.

24. A cell, tissue, or plant part of the sudangrass hybrid plant of claim 22.

25. A tissue culture of regenerable cells of the sudangrass hybrid plant of claim 22.

26. The tissue culture of claim 25, wherein cells of the tissue culture are produced from a tissue selected from the group consisting of leaf, stem, pollen, embryo, root tip, anther and silk.

27. Preserved forage of the sudangrass hybrid plant of claim 22.

28. The preserved forage of claim 27, wherein the preserved forage is hay, haylage, or silage.

29. A bale comprising the sudangrass hybrid plant of claim 22.

30. A pasture comprising the sudangrass hybrid plant of claim 22.

31. A tissue culture of regenerable cells, wherein the cells regenerate plants having all the morphological and physiological characteristics of the sudangrass hybrid plant of claim 22, representative seed of said hybrid having been deposited under ATCC Accession No. PTA-7193.

32-52. (canceled)
